(19) United States
(12) Patent Application Publication
(10) Pub. No.: US 2012/0329667 A1
(43) Pub. Date: Dec. 27, 2012

(54) EPIGENETIC DNA ENRICHMENT

(75) Inventors: Richard Allman, Victoria (AU); Eduardo Vom, Victoria (AU); Craig Matthew Lewis, Victoria (AU); Debbie Mantzaris, Victoria (AU); Stuart Cantsilieris, Victoria (AU)

(73) Assignee: GENETIC TECHNOLOGIES LIMITED, Victoria (AU)

(21) Appl. No.: 13/501,799

(22) PCT Filed: Oct. 13, 2010
(86) PCT No.: PCT/AU10/01345

§ 371 Date: Aug. 27, 2012

Related U.S. Application Data
(60) Provisional application No. 61/251,523, filed on Oct. 14, 2009.

(30) Foreign Application Priority Data
Oct. 14, 2009 (AU) 2009905023

(51) Int. Cl. C12Q 1/68; C07H 21/04; C40B 30/04; C12P 19/34

(52) U.S. Cl. 506/9; 435/91.5; 435/91.1; 536/23.1; 435/6.1; 435/6.14; 435/6.12

(57) ABSTRACT

This application relates to methods for enriching DNA from a first cell type from a sample comprising DNA from the first cell type and DNA from a second cell type, wherein the first cell type methylates DNA to a lesser extent than the second cell type. DNA is enriched by, selecting for fragments by size after digestion of the sample with a methylation-sensitive restriction enzyme. Cell types of interest include fetal cells and cancerous cells. The enriched DNA can be used for a variety of procedures including, detection of a trait of interest such as a disease trait, or a genetic predisposition thereto, gender typing and parentage testing.

### FIELD OF THE INVENTION

The present invention relates to methods for enriching DNA from a first cell type from a sample comprising DNA from the first cell type and DNA from a second cell type, wherein the first cell type methylates DNA to a lesser extent than the second cell type. The enriched DNA can be used for a variety of procedures including, detection of a trait of interest such as a disease trait, or a genetic predisposition thereto, gender typing and parentage testing.

### BACKGROUND OF THE INVENTION

The separation of individual components of a DNA mixture is not a trivial task. Much progress has been made in the deconvolution of mixed str profiles for forensic analysis. However, where more precise measurements of allelic ratios are required for medical diagnostic purposes, it is desirable that the component of interest is enriched as much as possible prior to genetic analysis. The applications of such mixture deconvolution are particularly evidenced in the analysis of tumour derived DNA samples (including circulating cell-free tumour DNA), and in prenatal genetic diagnosis for the enrichment of fetal DNA from that of the mother (including circulating cell-free fetal DNA).

In the case of prenatal genetic diagnosis the ratio of fetal:maternal DNA (from all sample types) is such that differentiation of subtle genetic differences between mother and child are extremely difficult. This is generally true for Mendelian genetic disorders involving point mutations or those instances where both parents are carriers for the same disease allele, as well as for the analysis of DNA polymorphisms that could be used to determine fetal ploidy.

Various techniques have been described in the art for the enrichment of different cell populations from a mixture, including fluorescence activated cell sorting (FACS), magnetic activated cell sorting, laser micro-dissection, micro-manipulation, immuno-affinity chromatography, differential centrifugation, density gradient centrifugation. Many of these methods rely upon the labelling of cell populations with a reagent, in particular monoclonal antibodies or DNA aptamers, to enable positive selection of the cells of interest, or negative selection whereby unwanted cells are labelled and removed. Both positive and negative selection may be achieved by direct (where a single reagent is utilised) or indirect (where a secondary detection reagent is required) means. All of the above methods are known in the art and have been applied to the sorting of cell subpopulations, including the detection and isolation of rare subpopulations such as stem cells, circulating tumour cells and circulating fetal cells.

More recently cell-free circulating nucleic acids have elicited much interest as potential molecular diagnostic tools in fields as diverse as cancer, stroke, trauma, myocardial infarction, autoimmune disorders, and prenatal diagnostics. Many of the current applications require detection of specific mutations, and thus do not require physical separation of DNA components. However, where the DNA component of interest is at a low concentration relative to the other component of the mixture some analyses become impossible without at least some degree of enrichment.

In general there have been two broad approaches to identifying/enriching fetal DNA from a mixed fetal/maternal source. One relies on the mild enrichment of cell-free circulating fetal DNA based upon the observation that most of the fetal DNA is highly fragmented and much of the large molecular weight maternal DNA can be removed by size selection on agarose gel (Li et al., 2004 and 2005; US 20080071076). The other relies on the identification of specific markers within fetal DNA which are differentially methylated between fetal and maternal DNA. The two most widely published being differences in the promoter regions of two tumour suppressor genes; Maspin (Serpin B5) which is methylated in maternal blood and unmethylated in circulating fetal DNA, and Rassf1a which is fully methylated in circulating fetal DNA and unmethylated in maternal blood DNA (U.S. Pat. No. 6,927,028; US 20090053719; US 20090155776; WO 2009/030100).

There is a need for alternate methods of enriching DNA from a sample comprising DNA from different sources.

### SUMMARY OF THE INVENTION

In one aspect, the present invention provides method of enriching fetal DNA from a sample comprising fetal DNA and maternal DNA, the method comprising

i) cleaving the DNA in the sample with a methylation sensitive restriction enzyme to produce a population of DNA fragments, and

ii) selecting DNA fragments which are less than about 200 kbp in size.

In an embodiment of the above aspect, the sample is, or is derived from, maternal blood, cervical mucous, a transcervical sample, a pap smear, or urine.

In another embodiment, the method further comprises enriching the sample for fetal cells, and extracting DNA from the cells before step i). The fetal cells can be enriched by any method known in the art including, but not limited to, by positive selection, negative selection, cell size, cell density, differential lysis, and/or charge flow separation.

In a further aspect, the present invention provides a method of enriching DNA from cancerous cells from a sample comprising DNA from cancerous and normal cells, the method comprising

i) cleaving the DNA in the sample with a methylation sensitive restriction enzyme to produce a population of DNA fragments, and

ii) selecting DNA fragments which are less than about 200 kbp in size.

The methods of the invention can be applied to any mixture comprising DNA from two different cell types that have different levels of DNA methylation. Thus, in a further aspect the present invention provides a method of enriching DNA from a first cell type from a sample comprising DNA from the first cell type and DNA from a second cell type, the method comprising

i) cleaving the DNA in the sample with a methylation sensitive restriction enzyme to produce a population of DNA fragments, and

ii) selecting DNA fragments which are less than about 200 kbp in size, wherein the first cell type methylates DNA to a lesser extent than the second cell type.

In one embodiment, the first cell type is a fetal cell and the second cell type is a maternal cell. In another embodiment, the first cell type is a cancer cell and the second cell type is a normal cell. In another embodiment, the first cell type is a transformed cell line and the second cell type is a normal cell. In another embodiment, the first cell type is a viral infected cell and the second cell type is the same cell type which is not infected with the virus.

In a further embodiment, DNA fragments which are less than about 150 kbp, less than about 100 kbp, less than about 50 kbp, less than about 30 kbp, less than about 20 kbp, less than about 15 kbp, or less than about 10 kbp, in size are selected. In a further embodiment, the selected DNA fragments are also greater than about 500 bp, greater than about 300 bp, or greater than about 100 bp, in size. In a preferred embodiment, DNA fragments which are less than about 30 kbp in size are selected. In a further preferred embodiment, DNA fragments between about 30 kbp and about 300 bp in size are selected.

Examples of methylation sensitive restriction enzymes which can be used for the invention include, but are not limited to, AatII, AciI, AclI, AfeI, AgeI, AscI, AsiSI, AvaI, BceAI, BmgBI, BsaAI, BsaHI, BsiEI, BsiWI, BsmBI, BspDI, BsrFI, BssHII, BstBI, BstUI, ClaI, EagI, FauI, FseI, FspI, HaeII, HgaI, HhaI, HinPII, HpaII, HpyChIV4, Hpy99I, KasI, MluI, NaeI, NarI, NgoMIV, NotI, NruI, PaeR7I, PmiI, PvuI, PsrII, SacII, SalI, SfoI, SgrAI, SmaI, SnaBI, TspMI, ZraI, or a combination of two or more thereof.

In a preferred embodiment, the methylation sensitive restriction enzyme has a adenine (A) and a thymine (T) within their recognition sequence.

In an embodiment, step ii) comprises separating the population of DNA fragments on an agarose gel, excising the portion of the gel comprising DNA fragments which are less than about 200 kbp, more preferably less than 30 kbp, more preferably less than 15 kbp, in size, and extracting the DNA fragments which are less than about 200 kbp, more preferably less than 30 kbp, more preferably less than 15 kbp, in size from the gel.

In a further embodiment, the method further comprises obtaining the sample.

Also provided is an enriched population of DNA fragments obtained by a method of the invention.

In another aspect, the present invention provides a composition comprising the DNA fragments of the invention, and a carrier.

Fetal DNA enriched using a method of the invention can be used to analyse the genotype of the fetus. Thus, in another aspect the present invention provides a method for analysing the genotype of a fetus at a locus of interest, the method comprising

i) obtaining enriched fetal DNA fragments using a method of the invention, and

ii) analysing the genotype of at least one of the fetal DNA fragment at a locus of interest.

In another aspect, the present invention provides a method for analysing the genotype of a fetus at a locus of interest, the method comprising

i) cleaving DNA in a sample comprising fetal DNA and maternal DNA with a methylation sensitive restriction enzyme to produce a population of DNA fragments,

ii) separating the DNA fragments based on size, and

iii) analysing the genotype of at least one fetal DNA fragment which is less than about 200 kbp in size at a locus of interest.

The genotype of the fetus can be determined using any suitable technique known in the art. Examples include, but are not limited to, hybridization based procedures, and/or amplification based procedures.

The genotype of a fetal DNA can be analysed for any purpose. Typically, the genotype will be analysed to detect the likelihood that the offspring will possess a trait of interest. Preferably, the fetal DNA is analysed for a genetic abnormality linked to a disease state, or predisposition thereto. In one embodiment, the genetic abnormality is in the structure and/or number or chromosomes. In another embodiment, the genetic abnormality encodes an abnormal protein. In another embodiment, the genetic abnormality results in decreased or increased expression levels of a gene.

The enriched fetal DNA can be used to determine the sex of the fetus. Thus, in a further aspect the present invention provides a method of determining the sex of a fetus, the method comprising

i) obtaining enriched fetal DNA fragments using a method of the invention, and

ii) analysing at least one of the fetal DNA fragments to determine the sex of the fetus.

In another aspect, the present invention provides a method of determining the sex of a fetus, the method comprising

i) cleaving DNA in a sample comprising fetal DNA and maternal DNA with a methylation sensitive restriction enzyme to produce a population of DNA fragments,

ii) separating the DNA fragments based on size, and

iii) analysing at least one of the fetal DNA fragments which is less than about 200 kbp in size to determine the sex of the fetus.

The analysis of the fetal DNA to determine the sex of the fetus can be performed using any technique known in the art. For example, Y-chromosome specific probes can be used.

The enriched fetal DNA can also be used to identify the father of the fetus. Accordingly, in a further aspect the present invention provides a method of determining the father of a fetus, the method comprising

i) obtaining enriched fetal DNA fragments using a method of the invention,

ii) determining the genotype of the fetus at one or more loci by analysing at least one of the fetal DNA fragments,

iii) determining the genotype of the candidate father at one or more of said loci, and

iv) comparing the genotypes of ii) and iii) to determine the probability that the candidate father is the biological father of the fetus.

In a further aspect, the present invention provides a method of determining the father of a fetus, the method comprising

i) cleaving DNA in a sample comprising fetal DNA and maternal DNA with a methylation sensitive restriction enzyme to produce a population of DNA fragments,

ii) separating the DNA fragments based on size,

iii) determining the genotype of the fetus at one or more loci by analysing at least one of the fetal DNA fragments which is less than about 200 kbp in size,

iv) determining the genotype of the candidate father at one or more of said loci, and

v) comparing the genotypes of iii) and iv) to determine the probability that the candidate father is the biological father of the fetus.

The methods of the invention can also be used to determine whether fetal cells are present in a sample, or DNA derived therefrom. Thus, in another aspect the present invention provides a method of detecting fetal DNA in a sample from a pregnant female, the method comprising

i) cleaving DNA in the sample obtained from the female with a methylation sensitive restriction enzyme to produce a population of DNA fragments, and

ii) comparing the amount of DNA fragments which are less than about 200 kbp in size produced in step i) with the amount of DNA fragments of the same size produced by cleaving the same amount of DNA from normal adult cells with the methylation sensitive restriction enzyme,

wherein a higher amount of DNA fragments which are less than about 200 kbp in size produced in step i) when compared to the amount of DNA fragments of the same size produced by cleaving the same amount of DNA from normal adult cells indicates the presence of fetal DNA in the sample.

In an embodiment of the above aspect, the sample is, or is derived from, maternal blood, cervical mucous, a transcervical sample, a pap smear, or urine.

The methods of the invention can also be used to determine whether cancerous cells are present in a sample, or DNA derived therefrom. Therefore, in another aspect the present invention provides a method of diagnosing and/or prognosing a cancer in a subject, the method comprising

i) cleaving DNA in a sample obtained from the subject with a methylation sensitive restriction enzyme to produce a population of DNA fragments, and

ii) comparing the amount of DNA fragments which are less than about 200 kbp in size produced in step i) with the amount of DNA fragments of the same size produced by cleaving the same amount of DNA from normal cells, preferably non-cancerous cells from the subject or non-cancerous cells of the same cell type from another subject, with the methylation sensitive restriction enzyme,

wherein a higher amount of DNA fragments which are less than about 200 kbp in size produced in step i) when compared to the amount of DNA fragments of the same size produced by cleaving the same amount of DNA from normal cells is diagnositic and/or prognostic of a cancer.

In a further aspect, the present invention provides a kit for enriching DNA from a first cell type from a sample comprising DNA from a second cell type, wherein the first cell type methylates DNA to a lesser extent than the second cell type, the kit comprising one or more methylation sensitive restriction enzymes.

In an embodiment, the kit further comprises one or more of the following;
i) an apparatus for obtaining the sample,
ii) an apparatus and/or media for transporting and/or storing the sample to a diagnostic laboratory,
iii) an apparatus for obtaining a second sample comprising maternal DNA but no fetal DNA from the mother,
iv) at least one reagent for extracting DNA from the sample, and
v) at least one reagent for performing a genetic assay.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—Agarose gel separation of adult and placental DNA following cleavage with HpaII. Moving left to right, Lanes 1 and 5 are DNA size markers, Lane 2 is adult female DNA, Lane 3 is adult male DNA and Lane 4 is placental DNA.

FIG. 2—Confirmation that fetal DNA standard performs well in the multiplexed STR analysis following restriction digest with HpaII or Eag1 restriction enzymes.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, fetal cell biology, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the term about, unless stated to the contrary, refers to +/−20%, more preferably +/−10%, of the designated value.

As used herein, the terms “enriching” and “enriched” are used in their broadest sense to encompass the isolation of DNA fragments derived from the first cell type (for example, fetal cells) such that the relative concentration of DNA fragments derived from the first cell type to DNA fragments derived from the second cell type is greater than a comparable untreated sample (before selection of the DNA fragments based on size). Preferably, the enriched DNA fragments derived from the first cell type are separated from at least 10%, more preferably at least 20%, more preferably at least 30%, more preferably at least 40%, more preferably at least 50%, more preferably at least 60%, more preferably at least 70%, more preferably at least 75%, more preferably at least 80%, more preferably at least 90%, more preferably at least 95%, and even more preferably at least 99% of the other DNA fragments. Most preferably, the enriched population contains no DNA fragments from the second cell type (namely, pure). The terms “enrich” and variations thereof are used interchangeably herein with the term “isolate” and variations thereof. Furthermore, a population of DNA fragments enriched using a method of the invention may only comprise a single DNA fragment.

As used herein, the term “fetal DNA” means any DNA directly or indirectly derived from the developing zygote, embryo or fetus and includes DNA from placental cells (trophoblasts) derived from the fetus. Similarly, the term “fetal cells” includes placental cells (trophoblasts) derived from the fetus.

As used herein, the term “diagnosis”, and variants thereof such as, but not limited to, “diagnose”, “diagnosed” or “diagnosing” includes any primary diagnosis of a clinical state or diagnosis of recurrent disease.

“Prognosis”, “prognosing” and variants thereof as used herein refer to the likely outcome or course of a disease, including the chance of recovery or recurrence.

### Methylation Sensitive Restriction Enzymes

DNA methylation is a covalent modification of DNA catalysed by DNA methyltransferase enzymes. Vertebrate methylation is dispersed over much of the genome, a pattern referred to as global methylation. In vertebrate genomes, the addition of a methyl group occurs exclusively on the cytosine within CG dinucleotides (referred to as CpG). Up to 90% of all CpGs are methylated in mammals (Bird, 1986). The exceptions are CpG islands, which are CpG enriched regions that frequently coincide with gene promoter regions at the 5′ ends of human genes and tend to be unmethylated (Bird, 1987). Because of this, the presence of a CpG island is used to help in the prediction and annotation of genes. Methylation of CpG sites within the promoters of genes can lead to their silencing, a feature found in a number of human cancers (for example the silencing of tumour suppressor genes), but also in the normal epigenetic control of genes and in so-called imprinted genes.

Restriction enzymes cleave both strands of a double-stranded DNA molecule, such as genomic DNA, at specific recognitions sequences. The number and size of fragments generated by a restriction enzyme depend on the frequency of occurrence of the target site in the DNA to be cut. Assuming a DNA molecule with a 50% G+C content and a random distribution of the four bases, a 4-base recognition site occurs every 4⁴ (256) bps. Similarly a 6-base recognition site occurs every 4⁶ (4096) bps, and a 8-base recognition site occurs every 4⁸ (65,536) bps. In practice, there is not a random distribution of the four bases and human DNA has approximately 43% G+C content.

The activity of many restriction enzymes is known to be influenced by DNA methylation. Examples of methylation sensitive restriction enzymes useful for the invention include, but are not limited to, those provided in Table 1.

As noted above, CpG enriched regions tend to be unmethylated. Therefore, in order to preferentially digest non-CpG island sites the preferred methylation sensitive restriction enzymes for use in the invention are those which incorporate A and T within their recognition sequence (for example, HPYChIV4, AcII, ClaI).

TABLE 1
Methylation sensitive restriction enzymes.

| Restriction | Number of bases | |
|---|---|---|
| Enzyme | in recognition site | Recognition sequence* |
| AatII | 6 | 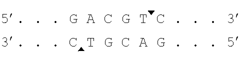 |
| AciI | 4 | 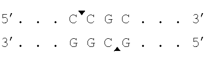 |
| AclI | 6 | 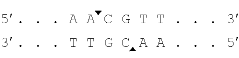 |
| AfeI | 6 | 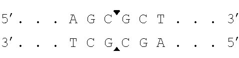 |
| AgeI | 6 | 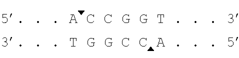 |
| AscI | 8 | 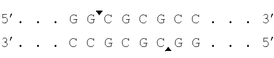 |
| AsiSI | 8 | 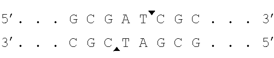 |
| AvaI | 6 | 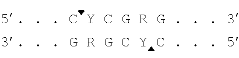 |
| BceAI | 6 | 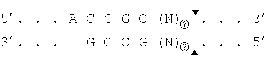 |
| BmgBI | 6 | 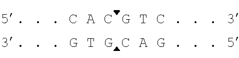 |
| BsaAI | 6 | 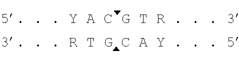 |
| BsaHI | 6 | 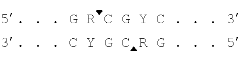 |
| BsiEI | 6 | 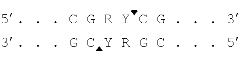 |
| BsiWI | 6 | 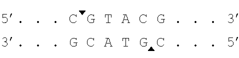 |
| BsmBI | 6 | 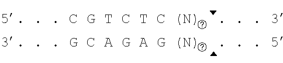 |
| BspDI | 6 | 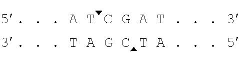 |
| BsrFI | 6 | 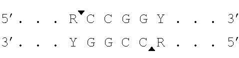 |
| BssHII | 6 | 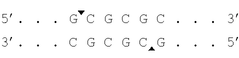 |
| BstBI | 6 | 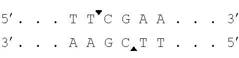 |
| BstUI | 4 | 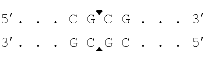 |
| ClaI | 6 | 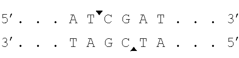 |
| EagI | 6 | 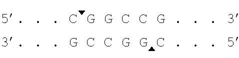 |
| FauI | 6 | 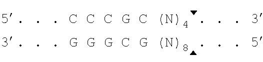 |
| FseI | 8 | 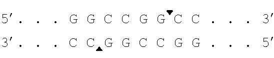 |
| FspI | 6 | 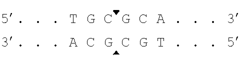 |
| HaeII | 6 | 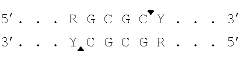 |
| HgaI | 6 | 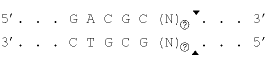 |
| HhaI | 4 | 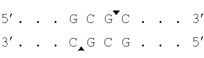 |
| HinP1I | 4 | 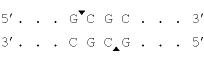 |
| HpaII | 4 | 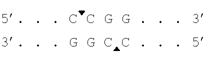 |
| HpyChIV4 | 4 | 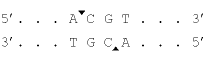 |
| Hpy99I | 5 | 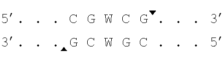 |
| KasI | 6 | 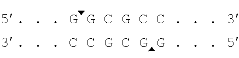 |
| MluI | 6 | 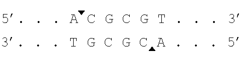 |
| NaeI | 6 | 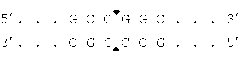 |
| NarI | 6 | 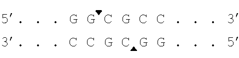 |
| NgoMIV | 6 | 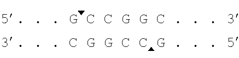 |
| NotI | 8 | 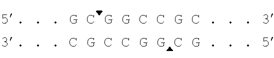 |
| NruI | 6 | 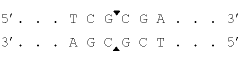 |
| PaeR7I | 6 | 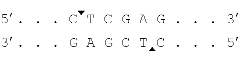 |
| PmlI | 6 | 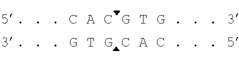 |
| PvuI | 6 | 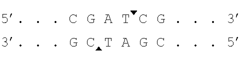 |
| RsrII | 7 | 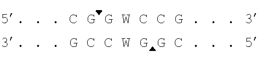 |
| SacII | 6 | 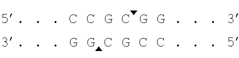 |
| SalI | 6 | 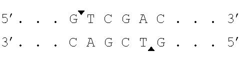 |
| SfoI | 6 | 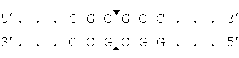 |
| SgrAI | 8 | 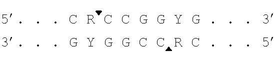 |
| SmaI | 6 | 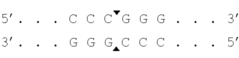 |
| SnaBI | 6 | 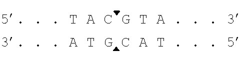 |
| TspMI | 6 | 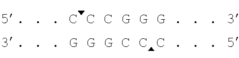 |
| ZraI | 6 | 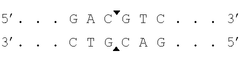 |

*B = C or G or T,
D = A or G or T,
H = A or C or T,
K = G or T,
M = A or C,
N = A or C or G or T,
R = A or G,
S = C or G,
V = A or C or G,
W = A or T and
Y = C or T.

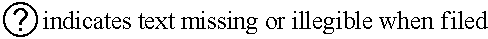

As the skilled person will appreciate, the restriction enzymes listed in Table 1 are readily available from commercial sources such as Promega and New England Biolabs. Cleavage will typically be performed in accordance with the manufacturer's instructions.

With regard to the phrase “wherein the first cell type methylates DNA to a lesser extent than the second cell type”, it is preferred that DNA from the first cell type comprises less than 10%, more preferably less than 25%, and more preferably less than 50%, methylated cytosines than DNA from the second cell type.

### Sample

The sample can be any biological sample which comprises a mixture of at least two different cell types with different levels of methylation, DNA derived from said cells, or a combination thereof. The nature of the sample will be dictated by the source of the DNA to be enriched and/or identified. The sample can comprise as little as one cell of the first cell type, or DNA derived therefrom.

As used herein, when referring to the sample, the phrase “derived from” means that there as been at least some human intervention changing the nature of the sample, typically at least partially purifying the cells and/or DNA from the biological sample, and/or extracting DNA therefrom.

Typically, the sample will be obtained from an organism with most of the DNA within intact cells. In these circumstances, it is preferred that the sample is at least partially processed to liberate the DNA from the cells. Techniques for processing samples to isolate DNA are well known in the art and include, but are not limited to, phenol/chloroform extraction (Sambrook et al., supra), QIAamp® Tissue Kit (Qiagen, Chatsworth, Calif), Wizard® Genomic DNA purification kit (Promega, Madison, Wis.), the A.S.A.P.™ Genomic DNA isolation kit (Boehringer Mannheim, Indianapolis, Ind.) and the Easy-DNA™ Kit (Invitrogen).

Before DNA extraction, the sample may also be processed to decrease the concentration of one or more sources of non-target DNA. In an embodiment, the sample is enriched for cells comprising target DNA. For example, when enriching for fetal DNA, the sample is first processed by positive or negative selection of fetal cells using known techniques, and then DNA extracted from the enriched cell population using one of the above-mentioned procedures.

In a preferred embodiment, the DNA is not treated such that it alters the chemical structure of the DNA in a manner that would effect cleavage with a methylation sensitive restriction enzyme. For example, the DNA is not treated with sodium bisulfate.

In an embodiment, the method comprises obtaining a biological sample (either directly from a subject or one which has previously been obtained from a subject), and extracting DNA from the sample before cleavage with the methylation sensitive restriction enzyme. In a further embodiment, the method comprises obtaining a biological sample (either directly from a subject or one which has previously been obtained from a subject), enriching the sample for cells of the first cell type, and extracting DNA from the sample before cleavage with the methylation sensitive restriction enzyme.

### Fetal Cells or DNA

Examples of the sources of biological material comprising fetal cells or DNA include, but are not limited to, blood, cervical mucous, a transcervical sample, a pap smear, or urine.

In a preferred embodiment, the sample is a transcervical sample. As used herein, the term “transcervical sample” refers to material taken directly from the pregnant female comprising cervical mucous. The transcervical sample can be obtained using a variety of sampling methods including, but not limited to, aspiration, irrigation, lavage and cell extraction. The sample may be obtained from sites including, but not limited to, the endocervical canal, external os, internal os, lower uterine pole and uterine cavity. A range of devices are available commercially which may be suitable for obtaining the sample, including but not limited to: “Aspiracath” aspiration catheter (Cook Medical, IN, USA), “Tao” brush endometrial sampler (Cook Medical, IN, USA), Goldstein Sonobiopsy catheter (Cook Medical, IN, USA), Aspiration kit (MedGyn, IL, USA), Endosampler (MedGyn, IL, USA), Endometrial sampler and cervical mucus sampling syringe (Rocket Medical, UK), “Sampling Probet” (Gynetics Products, Belgium), “Sampling in-out”—endometrial curette (Gynetics Products, Belgium), Endometrial cell sampler (Cheshire Medical Specialities Inc, CT, USA), Aspirette® Endocervical Aspirator and Embryo Transfer Catheter (Cooper Surgical, CT, USA), Intrauterine Catheter (Cooper Surgical, CT, USA), and the sampling device described in WO 2010/085841.

Once obtained, the sample comprising fetal cells is preferably stored at 0 to 4° C. until use. The sample is preferably transported and/or stored in HypoThermosol-FRS (HTS-FRS) Medium (Biolife Solutions) at 4° C. For long term storage, the sample is preferably stored in CryoStor CS5 (Biolife Solutions) at −80° C.

In a further embodiment, the sample comprising fetal cells is transported and/or stored in Gibco™ AmnioMaxII, Gibco™ AmnioMax C-100, or Gibco™ Keratinocyte-SFM supplemented with 2% fetal bovine serum, heparin (2500 U), hydrocortisone (5 μg/ml), insulin (5 μg/ml), human epidermal growth factor (5 μg/ml), human basic fibroblast growth factor (5 μg/ml), 25 μg/ml gentamycin, 50 ng/ml amphotericin B, 1-2 mmol/L vitamin C (ascorbic acid) or a water soluble analogue of vitamin E (1 mmol/L Trolox).

In one embodiment, the transport and/or storage media comprises serum such as bovine calf serum or human serum. In a further embodiment, the storage medium is degassed with nitrogen to reduce oxidative stress to the samples.

The methods of the invention for the enrichment of fetal DNA can be performed on any pregnant female of any mammalian species. Preferred mammals include, but are not limited to, humans, livestock animals such as sheep, cattle and horses, as well as companion animals such as cats and dogs.

The sample comprising fetal cells or DNA may be obtained at any stage of pregnancy. Preferably the sample is obtained during the first and second trimester of pregnancy. More preferably, the sample is obtained in the first trimester of pregnancy. Ideally the sample is obtained at a stage when a decision can be made for the well-being of the fetus and preferably within a period where an opportunity to make an early decision regarding therapeutic abortion can be made. Preferably, the sample is obtained up to 20 weeks of the pregnancy of a human female, more preferably within 5 to 20 weeks of pregnancy of a human.

In an embodiment, the method further comprises enriching the sample for fetal cells, in an embodiment at least enriching for trophoblasts. The fetal cells can be enriched by any method known in the art including, but not limited to, removal of non-cellular material, by positive selection, negative selection, cell size, cell density, differential lysis, and/or charge flow separation.

Fetal cell can be positively selected by using agents which bind molecules, typically proteins, which are not significantly produced by maternal cells in the sample. Examples of fetal cell markers include, but are not limited to, any molecule which is expressed by syncytiotrophoblasts and/or cytotrophoblasts but is not expressed by maternal cells. Examples include, but are not limited to, NDOG1 (AbCam, GeneTex, Serotec), NDOG2, Human Chorionic Gonadotropin (Calbiochem), MCP/cd46 (trophoblast/lymphocyte cross-reactive protein) (Abnova), TPBG (Trophoblast glycoprotein) (Abnova), GCSF receptor, ADFP (Adipose Differentiation Related Protein) (GenWay), Apolipoprotein H (AbCam), Placental Alkaline Phosphatase (AbCam), CXCR6 (Chemokine receptor 6) (R&D Systems), HLA-G (AbCam), CHL1 (extravillous cytotrophoblast antigen) (Abnova), Cytokeratin 7 (AbCam), Cytokeratin 8 (AbCam), Cytokeratin 18 (AbCam), FAS-Associated Phosphatase-1 (Leica), Folate Binding Protein (AbCam), FD0161G, Glucose Transporter GLUT3, H315, H316, HAI-1 (Hepatocyte growth factor activator protein-1 (EBioscience)), Human Placental Lactogen (Serotec), Id-1, Id-2, IBSP (Integrin Binding SialoProtein), MCSF-Receptor, MNF116, OKT9, plasminogen activator inhibitor 1 (AbCam), PLP-A (prolactin like proteins A) (Millipore Corporation), PLP-B (prolactin like proteins B), PLP-C (prolactin like proteins C), PLP-D (prolactin like proteins D), PLP-F (prolactin like proteins F), PLP-L (prolactin like proteins L), PLP-M (prolactin like proteins M), PLP-N (prolactin like proteins N), SP-1 (trophoblast specific beta 1 glycoprotein) (AbCam, BD Pharmingen), SSEA (Stage Specific Embryonic Antigen) (Novus Biologicals), TA1, TA2, Tfeb, Troma1, Trop1 (EBioscience) and Trop2, URO-4 (Adenosine Deaminase Binding Protein [ABP]) (Covance), or combination of any two or more thereof. Further methods of positively selecting fetal cells are described in WO 2009/103110.

As the skilled person will appreciate, negatively selecting fetal cells comprises removing from the sample cells that are identified/labelled as maternal. In other words, maternal cells are positively selected from the sample by targeting a molecule preferentially expressed in the maternal cells but not expressed in at least some fetal cells. In one embodiment, an agent (preferably an antibody) which binds at least one MHC molecule is used to select and remove maternal cells. Preferably, the agent binds an extracellular portion of the MHC molecule. As described in WO 2009/103110, methods for negatively selecting fetal cells based on differential MHC expression between fetal and maternal cells is known in the art. Other types of maternal cells that can be removed include, but are not limited to, maternal B cells, T cells, monocytes, macrophages, dendritic cells, vaginal epithelial cells, cervical epithelial cells, endometrial cells, maternal endothelial cells, maternal placental cells, polymorphs and mesenchymal cells of the placental villi each characterised by a specific set of surface markers that can be targeted for depletion. Examples of non-MHC molecules which can be targeted to possibly further deplete the sample of maternal cells include, but are not limited to, CD3, CD4, CD8, CD10, CD14, CD15, CD45, CD56 and proteins described by Blaschitz et al. (2000).

Methods for selecting fetal cells based on cell size are described in WO 2009/103110.

Density gradients may be used to enrich fetal cells, either as a single-step or multi-step procedure. Density gradients may be continuous or discontinuous and may be formed using media such as Metrizamide™, Ficoll and Percoll™. Further details of the use of density to enrich fetal cells are provided in WO 2004/076653.

Differential lysis exploits physical properties of cell membranes and, more particularly, cellular susceptibility to lysis in conditions different to the normal extracellular environment. In one embodiment, red blood cells may also be depleted by selective lysis using commercially available lysing solutions (eg, FACSlyse™, Becton Dickinson), Ammonium Chloride based lysing solutions or other osmotic lysing agents. In another embodiment, maternal cells bound by an antibody can be killed, and thus depleted from a sample, by complement-dependent lysis. For example, antibody labelled cells can be incubated with rabbit complement at 37° C. for 2 hr. Commercial sources for suitable complement systems include Calbiochem, Equitech-Bio and Pel Freez Biologicals. Suitable anti-MHC antibodies for use in complement-dependent lysis are known in the art, for example the W6/32 antibody (AbCam). Further details of the use of differential lysis to enrich fetal cells are described in WO 2004/076653.

Charge flow separation uses dielectrophoretic forces which occur on cells when a non-uniform electrical field interacts with field-induced electrical polarization. Depending on the dielectric properties of the cells relative to their suspending medium, these forces can be positive or negative, directing the cells toward strong or weak-electrical field regions. Because cells of different types or in distinct biological states have different dielectric properties, differential dielectrophoretic forces can be applied to drive their separation into purified cell populations (Wang et al., 2000).

### Cancerous Cells or DNA Therefrom

Essentially any biological material which comprises DNA from an organism which can get cancer, preferably a mammal, more preferably a human, can be used in the methods of the invention. Examples of such biological material include, but are not limited to, blood, plasma, serum, semen, bone marrow, urine or tissue biopsy.

Examples of tissue biopsies that can be used include, but are not limited to, from lung, kidney, liver, ovarian, head, neck, thyroid, bladder, cervical, colon, endometrial, esophageal, prostate or skin. Preferably, the tissue is suspected of comprising cancerous cells.

Methods for isolating a biological sample from a subject are known in the art and include, for example, surgery, biopsy, collection of a body fluid, for example, by paracentesis or thoracentesis or collection of, for example, blood or a fraction thereof. All such methods for isolating a biological sample shall be considered to be within the scope of providing or obtaining a biological sample.

For example, a cell or plurality of cells derived from a colorectum is collected or isolated using a method, such as, for example, a colonoscopy and/or collected from a stool sample. In the case of a sample from a prostate, the sample is collected, for example, by surgery (e.g., a radical prostatectomy) or a biopsy. In the case of a breast cancer, a sample is collected, for example, using a fine needle aspiration biopsy, a core needle biopsy, or a surgical biopsy.

### Selecting DNA Fragments

Typically, the selection of the DNA fragments will require the steps of

a) separating the DNA fragments based on size, and

b) isolating the DNA fragments of the desired size from the other DNA fragments.

The size separation of cleaved DNA can be brought about by a variety of methods, including but not limited to: chromatography or electrophoresis such as chromatography on agarose or polyacrylamide gels (Sambrook et al., supra), ion-pair reversed-phase high performance liquid chromatography (Hecker et al., 2000), capillary electrophoresis in a self-coating low-viscosity polymer matrix (Du et al., 2003), selective extraction in microfabricated electrophoresis devices (Lin et al., 2003), microchip electrophoresis on reduced viscosity polymer matrices (Xu et al., 2003), adsorptive membrane chromatography (Teeters et al., 2003), density gradient centrifugation (Raptis et al., 1980), and methods utilising nanotechnological means such as microfabricated entropic trap arrays (Han et al., 2002).

In one embodiment, the cleaved DNA is electrophoresed on an agarose gel (e.g. in the concentration range 0.5-2.0%). The DNA fragments of the desired size can then be isolated from the gel using commercially available kits (for example, Qiaex II supplied by Qiagen), by direct electro-elution, by centrifugation, or by any other method known in the art.

In one embodiment, the cleaved DNA is separated by centrifugation through a gel filtration medium (for example, Sephadex gel filtration columns).

### Analysis of Fetal DNA

Fetal DNA fragments isolated using the methods of the invention can be analysed for traits of interest and/or abnormalities of the fetus using techniques known in the art.

In one preferred embodiment, chromosomal abnormalities are detected. By “chromosomal abnormality” we include any gross abnormality in a chromosome or the number of chromosomes. For example, this includes detecting trisomy in chromosome 21 which is indicative of Down's syndrome, trisomy 18, trisomy 13, sex chromosomal abnormalities such as Klinefelter syndrome (47, XXY), XYY or Turner's syndrome, chromosome translocations and deletions, a small proportion of Down's syndrome patients have translocation and chromosomal deletion syndromes which include Pradar-Willi syndrome and Angelman syndrome, both of which involve deletions of part of chromosome 15, and the detection of mutations (such as deletions, insertions, transitions, transversions and other mutations) in individual genes. Other types of chromosomal problems also exist such as Fragile X syndrome, hemophilia, spinal muscular dystrophy, myotonic dystrophy, Menkes disease and neurofibromatosis, which can be detected by DNA analysis.

The phrase “genetic abnormality” also refers to a single nucleotide substitution, deletion, insertion, micro-deletion, micro-insertion, short deletion, short insertion, multinucleotide substitution, and abnormal DNA methylation and loss of imprint (LOI). Such a genetic abnormality can be related to an inherited genetic disease such as a single-gene disorder (e.g., cystic fibrosis, Canavan, Tay-Sachs disease, Gaucher disease, Familial Dysautonomia, Niemann-Pick disease, Fanconi anemia, Ataxia telengectasia, Bloom syndrome, Familial Mediterranean fever (FMF), X-linked spondyloepiphyseal dysplasia tarda, factor XI), an imprinting disorder [e.g., Angelman Syndrome, Prader-Willi Syndrome, Beckwith-Wiedemann syndrome, Myoclonus-dystonia syndrome (MDS)], or to predisposition to various diseases (e.g., mutations in the BRCA1 and BRCA2 genes). Other genetic disorders which can be detected by DNA analysis are known such as thalassaemia, Duchenne muscular dystrophy, connexin 26, congenital adrenal hypoplasia, X-linked hydrocephalus, ornithine transcarbamylase deficiency, Huntington's disease, mitochondrial disorder, mucopolysaccharidosis I or IV, Norrie's disease, Rett syndrome, Smith-Lemli Optiz syndrome, 21-hydroxylase deficiency or holocarboxylase synthetase deficiency, diastrophic dysplasia, galactosialidosis, gangliosidosis, hereditary sensory neuropathy, hypogammaglobulinaemia, hypophosphatasia, Leigh's syndrome, aspartylglucosaminuria, metachromatic leukodystrophy Wilson's disease, steroid sulfatase deficiency, X-linked adrenoleukodystrophy, phosphorylase kinase deficiency (Type VI glycogen storage disease) and debranching enzyme deficiency (Type III glycogen storage disease). These and other genetic diseases are mentioned in The Metabolic and Molecular Basis of Inherited Disease, 8th Edition, Volumes I, II, III and IV, Scriver, C. R. et al. (eds), McGraw Hill, 2001. Clearly, any genetic disease where the gene has been cloned and mutations detected can be analysed.

The methods of the present invention can also be used to determine the sex of the fetus. For example, staining of the isolated fetal DNA fragments with a Y-chromosome specific marker will indicate that the fetus is male, whereas the lack of staining will indicate that the fetus is female.

In yet another use of the invention, the methods described herein can be used for paternity testing. Where the paternity of a child is disputed, the procedures of the invention enable this issue to be resolved early on during pregnancy. Many procedures have been described for parentage testing which rely on the analysis of suitable polymorphic markers. As used herein, the phrase “polymorphic markers” refers to any nucleic acid change (e.g., substitution, deletion, insertion, inversion), variable number of tandem repeats (VNTR), short tandem repeats (STR), minisatellite variant repeats (MVR) and the like. Typically, parentage testing involves DNA fingerprinting targeting informative repeat regions, or the analysis of highly polymorphic regions of the genome such as HLA loci.

Diagnosis and/or Prognosis of Cancer

The present invention provides a method of diagnosing and/or prognosing cancer in a subject. In a preferred embodiment, the subject is a mammal In a particularly preferred embodiment, the subject is a human. Other preferred embodiments include companion animals such as cats and dogs, as well as livestock animals such as horses, cattle, sheep and goats.

The diagnostic and/or prognostic methods of the present invention involve a degree of DNA quantification which is readily provided by the inclusion of appropriate control samples from normal cells.

In one embodiment, internal controls are included in the methods of the present invention. A preferred internal control is one or more samples taken from one or more healthy individuals (also referred herein to as “normal cells”).

As will be known to those skilled in the art, when internal controls are not included in each assay conducted, the control may be derived from an established data set. Thus, in another embodiment, well defined standards which have been established as the result of previously analysing a sufficient numbers of samples for DNA from a particular cell type or source (e.g. tissue) are used for comparison with the test sample. Thus, it is not essential that a control sample be analysed when the test sample is analysed.

The control should comprise the same quantity of starting DNA as the sample to be analysed. As the skilled person will appreciate, there is some degree of flexibility in the term “same amount of DNA” as used herein because there will invariably be slight differences the actual amount of DNA. Preferably, this term means that the test sample and control sample have a DNA concentration which is no more than 10% difference in quantity.

In the present context, the term “healthy individual” shall be taken to mean an individual who is known not to suffer from cancer, such knowledge being derived from clinical data on the individual, including, but not limited to, a different diagnostic assay to that described herein.

Data pertaining to the control subjects are preferably selected from the group consisting of:

1. a data set comprising measurements of the amount of DNA fragments produced using the invention for a typical population of subjects known to have a cancer, or particular type of cancer;

2. a data set comprising measurements of the amount of DNA fragments produced using the invention for the subject being tested wherein said measurements have been made previously, such as, for example, when the subject was known to be healthy or, in the case of a subject having cancer, when the subject was diagnosed or at an earlier stage in disease progression;

3. a data set comprising measurements of the amount of DNA fragments produced using the invention for a healthy individual or a population of healthy individuals; and

4. a data set comprising measurements of the amount of DNA fragments produced using the invention for a normal individual or a population of normal individuals.

In the present context, the term “typical population” with respect to subjects known to have a cancer shall be taken to refer to a population or sample of subjects diagnosed with a cancer that is representative of the spectrum of the cancer patients. This is not to be taken as requiring a strict normal distribution of morphological or clinicopathological parameters in the population, since some variation in such a distribution is permissible. Preferably, a “typical population” will exhibit a spectrum of the cancer at different stages of disease progression.

As will be known to those skilled in the art, data obtained from a sufficiently large sample of the population will normalize, allowing the generation of a data set for determining the average amount of DNA fragments produced using the invention in normal cells/tissues of a given type.

Those skilled in the art are readily capable of determining the baseline for comparison in any diagnostic/prognostic assay of the present invention without undue experimentation, based upon the teaching provided herein.

### Analysis of DNA Fragments

DNA fragments enriched using the methods of the invention can be analysed by a variety of procedures, however, typically genetic assays will be performed. Genetic assay methods include the standard techniques of sequencing and PCR-based assays (including multiplex F-PCR STR analysis, QF-PCR, RT-PCR, and microarray analysis), as well as other methods described below.

The genetic assays may involve any suitable method for identifying mutations or polymorphisms, such as: sequencing of the DNA at one or more of the relevant positions; differential hybridisation of an oligonucleotide probe designed to hybridise at the relevant positions of either the wild-type or mutant sequence; denaturing gel electrophoresis following digestion with an appropriate restriction enzyme, preferably following amplification of the relevant DNA regions; S1 nuclease sequence analysis; non-denaturing gel electrophoresis, preferably following amplification of the relevant DNA regions; conventional RFLP (restriction fragment length polymorphism) assays; selective DNA amplification using oligonucleotides which are matched for the wild-type sequence and unmatched for the mutant sequence or vice versa; or the selective introduction of a restriction site using a PCR (or similar) primer matched for the wild-type or mutant genotype, followed by a restriction digest. The assay may be indirect, ie capable of detecting a mutation at another position or gene which is known to be linked to one or more of the mutant positions. The probes and primers may be fragments of DNA isolated from nature or may be synthetic.

A non-denaturing gel may be used to detect differing lengths of fragments resulting from digestion with an appropriate restriction enzyme. The DNA is usually amplified before digestion, for example using the polymerase chain reaction (PCR) method and modifications thereof.

Amplification of DNA may be achieved by the established PCR methods or by developments thereof or alternatives such as quantitative PCR, quantitative fluorescent PCR (QF-PCR), multiplex ligation dependent probe amplification, digital PCR, real time PCR (RT-PCR), single nuclei PCR, restriction fragment length polymorphism PCR (PCR-RFLP), PCR-RFLP/RT-PCR-RFLP, hot start PCR, nested PCR, in situ polonony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In another method, a pair of PCR primers are used which hybridise to either the wild-type genotype or the mutant genotype but not both. Whether amplified DNA is produced will then indicate the wild-type or mutant genotype (and hence phenotype).

A preferable method employs similar PCR primers but, as well as hybridising to only one of the wild-type or mutant sequences, they introduce a restriction site which is not otherwise there in either the wild-type or mutant sequences.

In order to facilitate subsequent cloning of amplified sequences, primers may have restriction enzyme sites appended to their 5′ ends. Thus, all nucleotides of the primers are derived from the gene sequence of interest or sequences adjacent to that gene except the few nucleotides necessary to form a restriction enzyme site. Such enzymes and sites are well known in the art. The primers themselves can be synthesized using techniques which are well known in the art. Generally, the primers can be made using synthesizing machines which are commercially available.

PCR techniques that utilize fluorescent dyes may also be used in the methods of the invention. These include, but are not limited to, the following five techniques.

i) Fluorescent dyes can be used to detect specific PCR amplified double stranded DNA product (e.g. ethidium bromide, or SYBR Green I).

ii) The 5′ nuclease (TaqMan) assay can be used which utilizes a specially constructed primer whose fluorescence is quenched until it is released by the nuclease activity of the Taq DNA polymerase during extension of the PCR product.

iii) Assays based on Molecular Beacon technology can be used which rely on a specially constructed oligonucleotide that when self-hybridized quenches fluorescence (fluorescent dye and quencher molecule are adjacent). Upon hybridization to a specific amplified PCR product, fluorescence is increased due to separation of the quencher from the fluorescent molecule.

iv) Assays based on Amplifluor (Intergen) technology can be used which utilize specially prepared primers, where again fluorescence is quenched due to self-hybridization. In this case, fluorescence is released during PCR amplification by extension through the primer sequence, which results in the separation of fluorescent and quencher molecules.

v) Assays that rely on an increase in fluorescence resonance energy transfer can be used which utilize two specially designed adjacent primers, which have different fluorochromes on their ends. When these primers anneal to a specific PCR amplified product, the two fluorochromes are brought together. The excitation of one fluorochrome results in an increase in fluorescence of the other fluorochrome.

### EXAMPLES

### Example 1

### Enrichment of Fetal DNA

DNA was obtained from adult males, adult females and placental cells (trophoblasts). The DNA was cleaved with HpaII and analysed on an agarose gel.

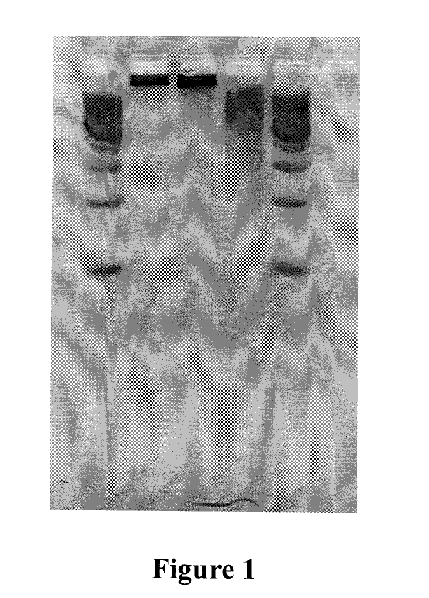

As shown in FIG. 1, cleavage of the placental DNA resulted in a much greater proportion of smaller DNA fragments than from cleavage of the adult DNA. As a result, a large proportion of the smaller fragments will be fetal in origin. These fragments can be selected and used for further analysis such as by QF-PCR using STR markers.

### Example 2

### Quantitative Fluorescent PCR Analysis of Enriched DNA

DNA was obtained from adult females and placental cells (trophoblasts). The DNA (100 ng) was cleaved with either HpaII or EagI restriction enzymes (1 U for 1 h at 37° C.). DNA was electrophoresed on agarose gel (0.8%) for 18 h at 10V. Lanes of interest were cut from the agarose gel and carefully inserted into dialysis tubing. DNA was then eluted from the gel with the short length of gel being perpendicular to the applied electric field for 30 min at 50V. This resulted in DNA fragments of less than about 25 kbp being selected.

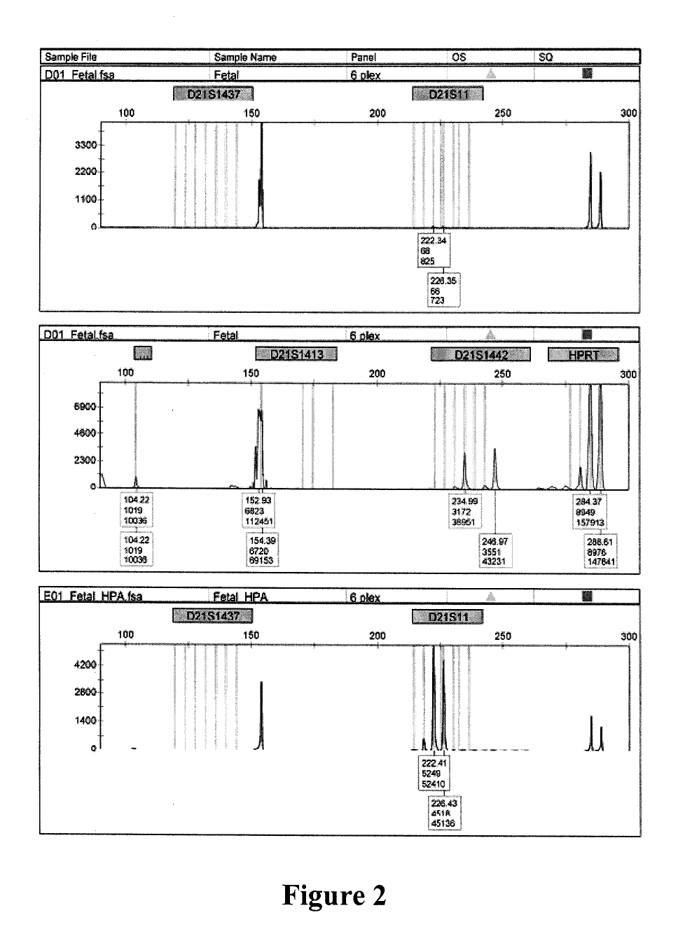

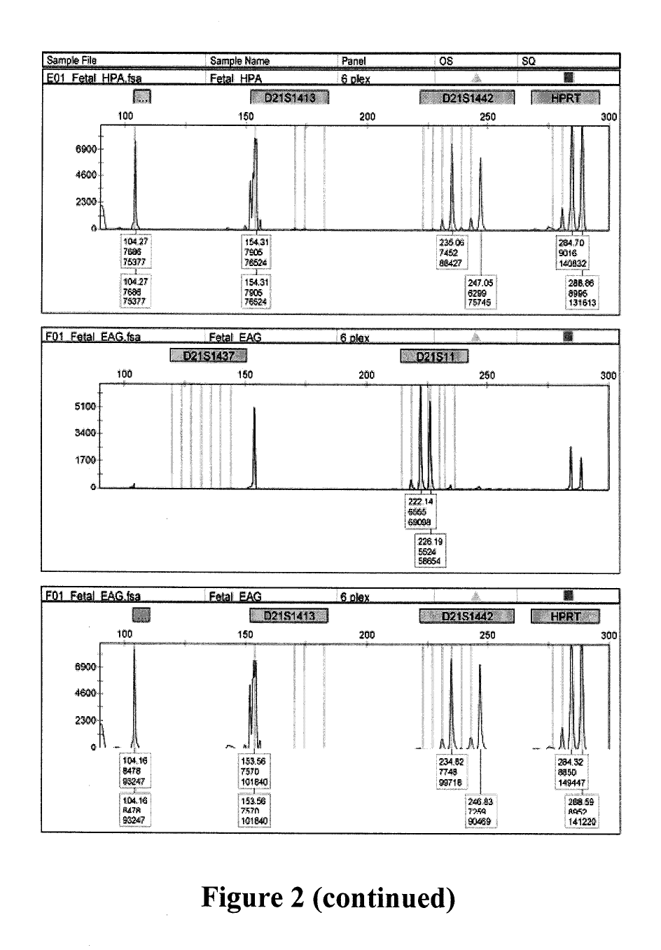

Eluted DNA was analysed using quantitative fluorescent PCR using a selection of chromosome 21 short tandem repeat (str) markers. Electrophoretograms indicate that the str-PCR was successful for DNA which had been cleaved with both restriction enzymes (FIG. 2).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from AU 2009905023 and US 61/251,523 both filed 14 Oct. 2009 and both of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Bird et al. (1986) Nature 321:209-213.
Bird et al. (1987) EMBO J. 6:999-1004.
Blaschitz et al. (2000) Placenta 21-733-741.
Du et al. (2003) Electrophoresis 24: 3147-3153.
Han et al. (2002) Analytical Chemistry 74: 394-401.
Hecker et al. (2000) Methods 46: 83-93.
Li et al. (2004) Clin. Chem. 50: 1002-1011.
Li et al. (2005) JAMA 293: 843-849.
Lin et al. (2003) J. Chromatogr. A. 1010: 255-268.
Raptis et al. (1980) J. Clin. Invest. 66: 1391-1399.
Teeters et al. (2003) J. Chromatogr. A. 989: 165-173.
Wang et al. (2000) Analytical Chemistry 72: 832-839.
Xu et al. (2003) Analyst. 128: 589-592.

1. A method of enriching fetal DNA from a sample comprising fetal DNA and maternal DNA, the method comprising
i) cleaving the DNA in the sample with a methylation sensitive restriction enzyme to produce a population of DNA fragments, and
ii) selecting DNA fragments which are less than about 200 kbp in size.

2. The method of claim 1, wherein the sample is, or is derived from, maternal blood, cervical mucous, a transcervical sample, a pap smear, or urine.

3. The method of claim 1 which further comprises enriching the sample for fetal cells, and extracting DNA from the cells before step i).

4. (canceled)

5. The method of claim 1, wherein the sample was obtained within 5 to 20 weeks of pregnancy of a human.

6. A method of enriching DNA from cancerous cells from a sample comprising DNA from cancerous and normal cells, the method comprising
i) cleaving the DNA in the sample with a methylation sensitive restriction enzyme to produce a population of DNA fragments, and
ii) selecting DNA fragments which are less than about 200 kbp in size.

7. (canceled)

8. A method of enriching DNA from a first cell type from a sample comprising DNA from the first cell type and DNA from a second cell type, the method comprising
i) cleaving the DNA in the sample with a methylation sensitive restriction enzyme to produce a population of DNA fragments, and
ii) selecting DNA fragments which are less than about 200 kbp in size,
wherein the first cell type methylates DNA to a lesser extent than the second cell type.

9. The method of claim 1, wherein DNA fragments which are less than about 30 kbp in size are selected.

10. The method of claim 1, wherein DNA fragments between about 30 kbp and about 300 bp in size are selected.

11. The method of claim 1, wherein the methylation sensitive restriction enzyme is selected from AatII, AciI, AclI, AfeI, AgeI, AscI, AsiSI, AvaI, BceAI, BmgBI, BsaAI, BsaHI, BsiEI, BsiWI, BsmBI, BspDI, BsrFI, BssHII, BstBI, BstUI, ClaI, EagI, FauI, FseI, FspI, HaeII, HgaI, HhaI, HinPII, HpaII, HpyChIV4, Hpy99I, KasI, MluI, NaeI, NarI, NgoMIV, NotI, NruI, PaeR7I, PmiI, PvuI, PsrII, SacII, SalI, SfoI, SgrAI, SmaI, SnaBI, TspMI, ZraI, or a combination of two or more thereof.

12. The method of claim 1, wherein step ii) comprises separating the population of DNA fragments on an agarose gel, excising the portion of the gel comprising DNA fragments which are less than about 200 kbp in size, and extracting the DNA fragments which are less than about 200 kbp in size from the gel.

13. (canceled)

14. An enriched population of DNA fragments obtained by the method of claim 1.

15. A composition comprising the DNA fragments of claim 14, and a carrier.

16. A method for analysing the genotype of a fetus at a locus of interest, the method comprising
i) obtaining enriched fetal DNA fragments using the method of claim 1, and
ii) analysing the genotype of at least one of the fetal DNA fragment at a locus of interest.

17. A method for analysing the genotype of a fetus at a locus of interest, the method comprising
i) cleaving DNA in a sample comprising fetal DNA and maternal DNA with a methylation sensitive restriction enzyme to produce a population of DNA fragments,
ii) separating the DNA fragments based on size, and
iii) analysing the genotype of at least one fetal DNA fragment which is less than about 200 kbp in size at a locus of interest.

18. (canceled)

19. A method of determining the sex of a fetus, the method comprising
i) obtaining enriched fetal DNA fragments using the method of claim 1, and
ii) analysing at least one of the fetal DNA fragments to determine the sex of the fetus.

20. A method of determining the sex of a fetus, the method comprising
i) cleaving DNA in a sample comprising fetal DNA and maternal DNA with a methylation sensitive restriction enzyme to produce a population of DNA fragments,
ii) separating the DNA fragments based on size, and
iii) analysing at least one of the fetal DNA fragments which is less than about 200 kbp in size to determine the sex of the fetus.

21. A method of determining the father of a fetus, the method comprising
i) obtaining enriched fetal DNA fragments using the method of claim 1,
ii) determining the genotype of the fetus at one or more loci by analysing at least one of the fetal DNA fragments,
iii) determining the genotype of the candidate father at one or more of said loci, and
iv) comparing the genotypes of ii) and iii) to determine the probability that the candidate father is the biological father of the fetus.

22. A method of determining the father of a fetus, the method comprising
i) cleaving DNA in a sample comprising fetal DNA and maternal DNA with a methylation sensitive restriction enzyme to produce a population of DNA fragments,
ii) separating the DNA fragments based on size,
iii) determining the genotype of the fetus at one or more loci by analysing at least one of the fetal DNA fragments which is less than about 200 kbp in size,
iv) determining the genotype of the candidate father at one or more of said loci, and
v) comparing the genotypes of iii) and iv) to determine the probability that the candidate father is the biological father of the fetus.

23. A method of detecting fetal DNA in a sample from a pregnant female, the method comprising
i) cleaving DNA in the sample obtained from the female with a methylation sensitive restriction enzyme to produce a population of DNA fragments, and
ii) comparing the amount of DNA fragments which are less than about 200 kbp in size produced in step i) with the amount of DNA fragments of the same size produced by cleaving the same amount of DNA from normal adult cells with the methylation sensitive restriction enzyme,
wherein a higher amount of DNA fragments which are less than about 200 kbp in size produced in step i) when compared to the amount of DNA fragments of the same size produced by cleaving the same amount of DNA from normal adult cells indicates the presence of fetal DNA in the sample.

24. (canceled)

25. A method of diagnosing and/or prognosing a cancer in a subject, the method comprising
i) cleaving DNA in a sample obtained from the subject with a methylation sensitive restriction enzyme to produce a population of DNA fragments, and
ii) comparing the amount of DNA fragments which are less than about 200 kbp in size produced in step i) with the amount of DNA fragments of the same size produced by cleaving the same amount of DNA from normal cells with the methylation sensitive restriction enzyme,
wherein a higher amount of DNA fragments which are less than about 200 kbp in size produced in step i) when compared to the amount of DNA fragments of the same size produced by cleaving the same amount of DNA from normal cells is diagnositic and/or prognostic of a cancer.

26. (canceled)

27. (canceled)

28. A kit for enriching DNA from a first cell type from a sample comprising DNA from a second cell type, wherein the first cell type methylates DNA to a lesser extent than the second cell type, the kit comprising one or more methylation sensitive restriction enzymes.
